(19) United States
(12) Patent Application Publication
(10) Pub. No.: US 2025/0248909 A1
(43) Pub. Date: Aug. 7, 2025

(54) COMPOSITION AND METHODS FOR TREATING AGING HAIR

(71) Applicant: CHL Industries, LLC, Frisco, TX (US)

(72) Inventors: John P. McCook, Frisco, TX (US); David B. Vasily, BETHLEHEM, PA (US)

(21) Appl. No.: 17/887,471

(22) Filed: Aug. 14, 2022

Related U.S. Application Data
(60) Provisional application No. 63/233,234, filed on Aug. 14, 2021.

(51) Int. Cl. A61K 8/19; A61Q 5/00; A61Q 5/06

(52) U.S. Cl. CPC A61K 8/19; A61Q 5/002; A61Q 5/065

(57) ABSTRACT

Methods for increasing the density and diameter of human hair comprised of the step of applying to any skin area of the body and/or the scalp covered with hair, a topical hair volumizing composition comprised of a chelated copper chlorin compound, wherein the chelated copper chlorin compound is comprised of at least 75% of a chlorin species selected from the group consisting of trisodium copper (II) chlorin e6 and disodium copper (II) isochlorin e4, and the chelated copper chlorin compound is present at a concentration of from 0.02 to 0.1% by weight of the composition.

### CROSS REFERENCE TO RELATED APPLICATIONS

This application claims priority under 35 U.S.C. § 119(e) to U.S. Provisional Application Ser. No. 63/233,234 (filed on Aug. 14, 2021), the disclosure of which is incorporated herein by reference.

### FIELD OF INVENTION

Topically applied compositions and methods for reducing the signs of human hair aging by increasing hair density.

### BACKGROUND OF THE INVENTION

Disease, genetics, chemical exposure, nutrition, environmental or other stressors (lifestyle), and the aging process itself contribute to reduction in hair density. There has been and remains a need for safe and effective topically applied compositions for treating aging hair that can increase both the number and diameter of terminal hairs. Those needs are met by the novel compounds, topically applied compositions containing those compounds and methods of the present invention.

### BRIEF DESCRIPTION OF DRAWINGS

FIGS. 1a and 1b are color photographs of Subject 001 at baseline and 90±10 days after once-daily application of a topical hair volumizing composition containing a chelated copper chlorin compound in accordance with methods of the present invention.

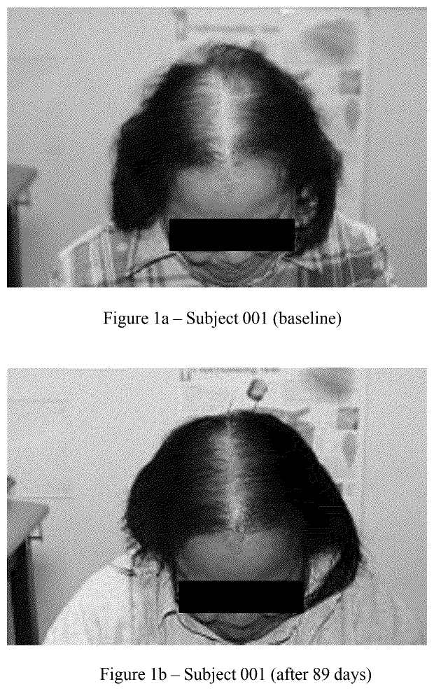

FIGS. 2a and 2b are color photographs of Subject 006 at baseline and 90±10 days after once-daily application of a topical hair volumizing composition containing a chelated copper chlorin compound in accordance with methods of the present invention.

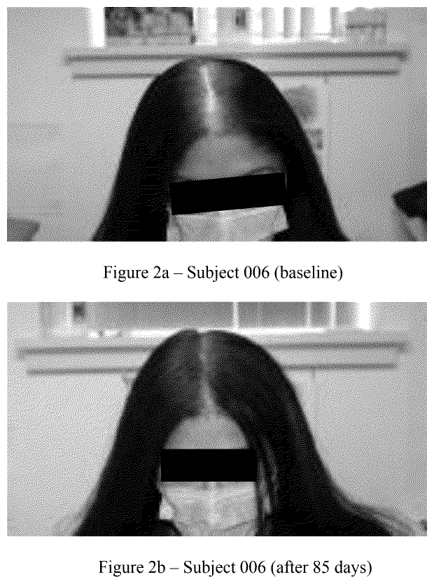

### SUMMARY OF THE INVENTION

Methods for increasing the density and diameter of human hair comprised of the step of applying to any skin area of the body and/or the scalp covered with hair, a topical hair volumizing composition comprised of, consisting essentially of, or consisting of a chelated copper chlorin compound, wherein the chelated copper chlorin compound is comprised of at least 75% of a chlorin species selected from the group consisting of trisodium copper (II) chlorin e6 and disodium copper (II) isochlorin e4, and the chelated copper chlorin compound is present at a concentration of from 0.02 to 0.1% by weight of the composition.

### DETAILED DESCRIPTION OF THE INVENTION

“At least one” means one or more and includes individual components as well as mixtures/combinations.

Numbers used in describing ingredients are to be understood as being modified in all instances by the term “about.”

Numerical ranges are meant to include numbers within the recited range, and combinations of subranges between, the given ranges. For example, a range from 1-5, includes 1, 2, 3, 4 and 5, as well as subranges such as 2-5, 3-5, 2-3, 2-4, 1-4, etc.

Unless otherwise indicated, percentages, parts and ratios are to be understood as based upon the total weight of the chelated copper chlorin compound or a topical hair volumizing composition containing the chelated copper chlorin compound.

“Terminal hair” has a diameter of 40 microns or greater.

“Vellus hair” has a diameter of less than 40 microns.

“Average terminal hair density” is the density of terminal hairs in a defined area.

“Average total hair density” is the density of terminal hairs and vellus hairs in a defined area.

A skin area of the body and/or the scalp covered with hair (including eyebrows, eyelashes, or beard) meeting one or more of the following criteria based on trichometric analysis of a measured area (e.g., 83.2 mm²) is considered “thinning” if. (i) the average (mean) hair diameter of at least 25% of terminal hairs in the measured area is less than 80 microns; (ii) the density of vellus hairs in the measured area is at least 15%; (iii) the ratio of terminal hairs to vellus hairs in the measured area is about 5:1; (iv) one standard deviation of the mean diameter of terminal hairs divided by the mean diameter of terminal hairs is 0.2 or greater.

Thinning hair may be visible and appear as a receding hairline and/or bald spots in men and thinning hair along the crown of the scalp in women. Thinning hair may, however, not be visible, but can be seen under magnification (hi-resolution photography, trichoscopy, or microscope).

Hair may also be characterized as “thinning” based on one or more of the following observations: perceived widening of the hair part; seeing more skin on the scalp (e.g., when hair is pulled back); more hair on a pillow; more hair strands on a brush; more hair in the drain; sunburn on the scalp; hair pull test (six or more stands fall out when about 40 strands are grasped from different parts of the scalp and gently tugged); hair tug test (hair breaks when a section of hair is grasped with two hands, one near the root and one near the tip, and tugged); a thinner ponytail or increased looping of the holder around the ponytail.

Disodium copper (II) isochlorin e4 is a porphyrin compound also known as disodium 3-[(2Z,4S,5S,16Z)-2-(carboxylatomethyl)-10-ethenyl-15-ethyl-5,9,14,19-tetramethyl-21,23,24,25-tetraaza-22-cuprahexacyclo[9.9.3.1\hat{ }{3,6}.1\hat{ }{13,16}.0\hat{ }{8,23}.0\hat{ }{18,21}]pentacosa-1(20),2,6(25),7,9,11,13(24),14,16,18-decaen-4-yl]propanoate. Neither a CAS number nor an EINECS number has been assigned to this compound.

Trisodium copper (II) chlorin e6 is a porphyrin compound and has been assigned CAS number 11006-34-1 and EINECS number 234-242-5.

Methods of the present invention comprise the step of applying a topical hair volumizing composition comprised of (or consisting essentially of) a chelated copper chlorin compound to any skin area of the body and/or the scalp covered with hair.

In certain preferred embodiments, the topical hair volumizing composition comprised of (or consisting essentially) of a chelated copper chlorin compound is applied to an area of “thinning” hair as defined above.

In a first set of embodiments, topical hair volumizing compositions used in practicing the methods of the present invention contain a chelated copper chlorin compound that is comprised of at least 75%, preferably at least about 80%, more preferably at least about 85%, still more preferably at least about 90%, even more preferably at least about 95% of disodium copper (II) isochlorin e4. In these compositions, disodium copper (II) isochlorin e4 is present at a concentration of from about 0.001% to about 0.5%, preferably from about 0.01% to 0.25%, and more preferably from about 0.02% to about 0.1%.

In a second set of embodiments, topical hair volumizing compositions used in practicing the methods of the present invention contain a chelated copper chlorin compound that is comprised of at least 75%, preferably at least about 80%, more preferably at least about 85%, still more preferably at least about 90%, even more preferably at least about 95% of trisodium copper (II) chlorin e6. In these compositions, trisodium copper (II) chlorin e6 is present at a concentration of from about 0.001% to about 0.5%, preferably from about 0.01% to 0.25%, and more preferably from about 0.02% to about 0.1%.

In a third set of embodiments, topical hair volumizing compositions used in practicing the methods of the present invention contain a chelated copper chlorin compound comprised of (a) at least 30% by weight of disodium copper (II) isochlorin e4 or trisodium copper (II) chlorin e6 and (b) at least one chlorin compound selected from the group consisting of chlorin e4; isochlorin e4; copper chlorin e4; copper isochlorin e4; chlorin e6; copper chlorin p6; copper pheophorbide a; copper pyropheophorbide a; copper purpurin 7; copper rhodin g7; copper rhodochlorin; and oxidized forms and salts of the above listed chlorin compounds.

In these embodiments, the chelated copper chlorin compound is present in the topical hair volumizing composition at a concentration of from about 0.001% to about 0.5%, preferably from about 0.01% to 0.25%, and more preferably from about 0.02% to about 0.1%.

In a fourth set of embodiments, the topical hair volumizing compositions used in practicing the methods of the present invention contain two chelated copper chlorin compounds: a first chelated copper chlorin compound comprised at least 75% by weight disodium copper (II) isochlorin e4 or trisodium copper (II) chlorin e6; and a second chelated copper chlorin compound comprised (a) at least 30% by weight of disodium copper (II) isochlorin e4 and (b) at least one chlorin compound selected from the group consisting of chlorin e4; isochlorin e4; copper chlorin e4; copper isochlorin e4; chlorin e6; copper chlorin p6; copper pheophorbide a; copper pyropheophorbide a; copper purpurin 7; copper rhodin g7; copper rhodochlorin; and oxidized forms and salts of the above listed chlorin compounds.

In these embodiments, the first and second chelated copper chlorin compounds are present in the topical hair volumizing composition at a combined concentration of from about 0.001% to about 0.5%, preferably from about 0.01% to 0.25%, and more preferably from about 0.02% to about 0.1%.

In practicing the methods of the present invention, the topical hair volumizing composition containing a chelated copper chlorin compound may be applied once or several times daily, or at different intervals (e.g., several days per week), at different doses. The topical hair volumizing composition containing a chelated copper chlorin compound may, for example, be applied; once every two days; once every three days; once every four days; once every five days; once every six days; once per week; over the course of a week, for two consecutive days followed by one, two, three, four or five “off” days; over the course of a week, for three consecutive days followed by one, two, three, or four “off” days; over the course of a week, for four consecutive days followed by one, two, or three “off” days; over the course of a week, for five consecutive days followed by one or two “off” days; over the course of a week, for six consecutive days followed by one “off” days; two days/week; three days/week; four days/week; five days/week; six days/week.

To achieve an increase of average density of terminal hairs of greater than 15%, a topical hair volumizing composition containing a chelated copper chlorin compound in accordance with the first, second, third or fourth embodiments of the present invention is preferably applied once daily for at least 70 days, preferably for at least 75 days, more preferably for at least 80 days, still more preferably for at least 85 days, in each case without skipping application on two consecutive days.

To achieve an increase of average diameter of terminal hairs of greater than 5%, a topical hair volumizing composition containing a chelated copper chlorin compound in accordance with the first, second, third or fourth embodiments of the present invention is preferably applied once daily for at least 70 days, preferably for at least 75 days, more preferably for at least 80 days, still more preferably for at least 85 days, in each case without skipping application on two consecutive days.

To achieve an increase of average density of terminal hairs of greater than 25%, a topical hair volumizing composition containing a chelated copper chlorin compound in accordance with the first, second, third or fourth embodiments of the present invention is preferably applied once daily for at least 150 days, preferably for at least 155 days, more preferably for at least 160 days, still more preferably for at least 165 days, even more preferably for at least 170 days, in each case without skipping application on two consecutive days.

To achieve an increase of average density of terminal hairs of greater than 25%, it is especially preferred that a topical hair volumizing composition containing a chelated copper chlorin compound in accordance with the first, second, third or fourth embodiments of the present invention is preferably applied once daily for at least 175 days, and even more preferably for at least 180 days, in each case without skipping application on two consecutive days.

To achieve an increase of average diameter of terminal hairs of greater than 15%, a topical hair volumizing composition containing a chelated copper chlorin compound in accordance with the first, second, third or fourth embodiments of the present invention is preferably applied once daily for at least 150 days, preferably for at least 155 days, more preferably for at least 160 days, still more preferably for at least 165 days, even more preferably for at least 170 days, in each case without skipping application on two consecutive days.

To achieve an increase of average diameter of terminal hairs of greater than 15%, it is especially preferred that a topical hair volumizing composition containing a chelated copper chlorin compound in accordance with the first, second, third or fourth embodiments of the present invention is preferably applied once daily for at least 175 days, and even more preferably for at least 180 days, in each case without skipping application on two consecutive days.

Dosing and frequency of methods of the present invention can and will vary depending on whether in addition to application of a topical hair volumizing composition containing a chelated copper chlorin compound one (or several) additional treatment step(s) is/are practiced concommitantly. Additional treatment steps may include one or a combination of (i) administering 6-piperidin-1-ylpyrimidine-2,4-diamine 3-oxide, (ii) administering a 5-α reductase inhibitor selected from the group of 4-azasteroid compounds, aldosterone receptor antagonists, (iii) administering a retinoid (defined below) (iv) injecting platelet-rich plasma, (v) injecting a corticosteroid, (vi) microneedling (defined below) or photobiostimulation by low-level laser (light) therapy (LLLT) or light emitting diodes (LED) as described in the following scientific publications, the disclosures of which are incorporated, in pertinent part, herein by reference: Avci, P. et al, in Lasers Surg Med. 2014 February; 46(2): 144-151; Leavitt M. et al, Clin Drug Investig. 2009; 29(5):283-292; Barolet D., Semin Cutan Med Surg. 2008; 27(4):227-238 Clin Drug Investig. 2009; 29(5):283-292.

“5-α reductase inhibitors” include 4-azasteroid compounds (finasteride and dutasteride) and aldosterone receptor antagonists (spironolactone).

“Retinoids” are a group of compounds including retinoic acid (RA), its active metabolites all-trans-retinol, Tretinoin (all-trans-retinoic acid), Isotretinoin (13-cis-retinoic acid), Alitretinoin (9-cis-retinoic acid), and can be non-aromatic, monoaromatic or contain a cyclic polyene side chain. Non-limiting examples include Retinyl Palmitate, Retinaldehyde, Tazarotene, Adapalene.

“Microneedling” (also known in the art as “dermarolling”) is a minimally invasive procedure involving the induction of percutaneous wounds by rolling a dermaroller—a circular array of microneedles (e.g., 0.5-3 mm in length and 0.1-0.25 mm in diameter) studded on drum-shaped cylinder (e.g., 192 microneedles in 8 rows, each row having 24 microneedles) over an area of skin, creating micropunctures in the stratum corneum.

Topical hair volumizing compositions used in practicing the methods of the present invention contain “carriers”—liquids and semi-solids—known to the person having ordinary skill in the art (e.g., alcohol, glycols, esters, silicones) in which the chelated copper chlorin compound is miscible or dispersible and can be administered in different dosage forms—spray, foam, cream, lotion, gel, serum. The composition can contain water (as a water-in-oil emulsion, an oil-in-water emulsion, a water-in-silicone emulsion, a silicone-in-water emulsion, a water-in-oil-in-water emulsion, or a thickened aqueous gel) or can be anhydrous.

Topical hair volumizing compositions used in practicing the methods of the present invention, can and in certain embodiments, do include one or more ingredients that provide a skin or hair “benefit” (e.g., reduce one or more of dryness, oiliness, visible redness, hair strand brittleness or breakage; provide hydration, barrier protection, and/or protection from ultraviolet radiation or other environmental stressors; impart shine or softness; improve combability; increase color vibrancy). Non-limiting examples of skin or hair “benefit” ingredients include antioxidants, anti-inflammatory agents, humectants, skin penetration enhancers (e.g., Ethoxydiglycol), over-the-counter or prescription active pharmaceutical ingredients (including skin protectant drug products, antimicrobial drug products, sunscreens), botanical or marine extracts, growth factors, peptides, amino acids.

Preferred, but non-limiting examples of ingredients, that can provide a skin or hair “benefit” include: 5-α reductase inhibitors (defined above); retinoids (defined above); bis-aminopropyl diglycol dimaleate; caffeine and theophylline; Curcuma longa extract; Withania somnifera extract; Serenoa serrulata fruit extract; resveratrol; ubiquinone; Camellia sinensis leaf extract; L-methionine; L-cysteine hydrochloride; pantothenic acid and its derivatives; biotin; selenium; zinc; cholecalciferol; sodium hyaluronate.

Non-limiting examples of anionic polysaccharides that can be, and in certain preferred embodiments, are cellulosic ethers; carrageenan; kelp; xanthan gum; tragacanth; gum acacia; locust bean gum.

Disodium copper (II) isochlorin e4 or trisodium copper (II) chlorin e6 are labile; over time, they are susceptible to oxidative, temperature-induced and other types of degradation indicated by change in color value (e.g., CIELAB color space). “Stabilization” of these compounds is demonstrated by reduction in change from initial color value. Accordingly, in preferred embodiments, the topical hair volumizing compositions of the present invention contains one or both of an anionic polysaccharide and/or an antioxidant in “stabilizing” amount(s) as measured by reduction in colorimetric change.

One preferred dosage form is a hydro-alcoholic spray administered through a trigger or pump sprayer, a pressurized aerosol sprayer, or a bag-on-valve sprayer.

### EXAMPLE

### Example A—Topical Hair Volumizing Composition

| Ingredient | % wt/wt |
|---|---|
| Chelated Copper Chlorin Compound | 0.02-0.1 |
| SD Alcohol, 190 proof | 30-40 |
| Butylene Glycol | 5-20 |
| Ethoxydiglycol (Diethylene Glycol Monoethyl Ether) | 2-10 |
| Phosphatidylcholine or Lecithin | 0.2-3 |
| Antioxidant | 0.01-0.1 |
| pH adjusting agent | 0.01-0.1 |
| Water | QS to 100% |

### Example B—Study

Seven otherwise healthy female adults meeting the following criterion were enrolled in a study with an initial planned duration of 6 months: one standard deviation of the mean diameter of terminal hairs divided by the mean diameter of terminal hairs of 0.2 or greater based trichoscopic imaging (TrichoSciencePro© with version 1.6 software; Trilogic LLC, Boston, MA) at 25× magnification within a measurement area of 82.3 sq. mm at either mid-parting area 3 cm from anterior hairline of frontal scalp or vertex mid-part section of scalp based on the area of maximum visible hair thinning.

Test subjects sprayed the topical hair volumizing composition of Example A to an area of the scalp exhibiting “thinning” at dose of 1 gram per 700 cm² once daily over a first study period of 90±10 consecutive days and continuing over a second study period of an additional 90±10 days. The following results were observed.

Among all seven subjects, average terminal hair density increased after 90 days±10 days by 18.7% (p<0.05; small sample t-test).

Among all seven subjects, average terminal hair density increased after 180 days±10 days by 29% (p<0.05; small sample t-test).

Among all seven subjects, average terminal hair diameter increased after 90 days±10 days by 6.3% (p<0.05; small sample t-test).

Among all seven subjects, average terminal hair diameter increased after 180 days±10 days by 16.2% (p<0.01; small sample t-test).

Among all seven subjects, average vellus hair density decreased after 90 days±10 days by 55.4% (p<0.01; small sample t-test).

Among all seven subjects, average vellus hair density decreased after 180 days±10 days by 73.8% (p<0.01; small sample t-test).

Among all seven subjects, average ratio of terminal hairs to vellus hairs were: 4.8:1 at baseline; 12.7:1 at 90 days, and 21.5:1 at 180 days.

TABLE 1
Average Terminal Hair Density

| Subject | Baseline | 90 days ± 10 days | 180 days ± 10 days |
|---|---|---|---|
| 001 | 28 | 44 | 56 |
| 002 | 38 | 54 | 54 |
| 003 | 65 | 66 | 70 |
| 004 | 38 | 42 | 47 |
| 005 | 46 | 55 | 50 |
| 006 | 44 | 50 | 50 |
| 007 | 52 | 58 | 67 |

TABLE 2
Average Terminal Hair Diameter

| Subject | Baseline | 90 days ± 10 days | 180 days ± 10 days |
|---|---|---|---|
| 001 | 76 | 82 | 89 |
| 002 | 79 | 76 | 77 |
| 003 | 100 | 103 | 121 |
| 004 | 69 | 70 | 83 |
| 005 | 78 | 88 | 98 |
| 006 | 76 | 82 | 80 |
| 007 | 77 | 89 | 97 |

TABLE 3
Average Vellus Hair Density

| Subject | Baseline | 90 days ± 10 days | 180 days ± 10 days |
|---|---|---|---|
| 001 | 6 | 0 | 11* |
| 002 | 11 | 7 | 0 |
| 003 | 5 | 0 | 0 |
| 004 | 7 | 4 | 0 |
| 005 | 12 | 5 | 0 |
| 006 | 11 | 6 | 4 |
| 007 | 13 | 7 | 2 |

*outlier; potential measurement error

The preceding examples illustrate various aspects and preferred embodiments of the invention but are not intended to be limiting in any way. While particular embodiments of the present invention have been illustrated and described, those skilled in the art will understand that changes and modifications can be made without departing from the spirit and scope of the invention. Accordingly, all changes and modifications that are within the scope of this invention are intended to be covered in the appended claims.

1. A method for increasing a density or a diameter of terminal hairs comprising a step of applying a topical hair volumizing composition comprising a carrier and 0.001% to 0.1% by weight of an active ingredient useful in increasing the density or the diameter of terminal hairs to an area of human scalp experiencing hair thinning resulting from aging;
wherein the active ingredient consists of one or more chelated copper chlorin compounds; and
wherein the one or more chelated copper chlorin compounds comprises at least 75% by weight total of a copper chlorin e6, a salt form of the copper chlorin e6, an oxidized form of the copper chlorin e6, or an oxidized form of the salt form of the copper chlorin e6 or a combination thereof.

2. The method of claim 1 wherein the one or more chelated copper chlorin compounds comprises the salt form of the copper chlorin e6 or the oxidized form of the salt form of the copper chlorin e6 or both; and
wherein the one or more chelated copper chlorin compounds further comprises one or more of:
(a) a copper chlorin e4, a salt form of the copper chlorin e4, an oxidized form of the copper chlorin e4, an oxidized form of the salt form of the copper chlorin e4, or a combination thereof,
(b) a copper isochlorin e4, a salt form of the copper isochlorin e4, an oxidized form of the copper isochlorin e4, an oxidized form of the salt form of the copper isochlorin e4, or a combination thereof,
(c) a copper chlorin p6, a salt form of the copper chlorin p6, an oxidized form of the copper chlorin p6, an oxidized form of the salt form of the copper chlorin p6, or a combination thereof,
(d) a copper pheophorbide a, a salt form of the copper pheophorbide a, an oxidized form of the copper pheophorbide a, an oxidized form of the salt form of the copper pheophorbide a, or a combination thereof,
(e) a copper pyropheophorbide a, a salt form of the copper pyropheophorbide a, an oxidized form of the copper pyropheophorbide a, an oxidized form of the salt form of the copper pyropheophorbide a, or a combination thereof,
(f) a copper purpurin 7, a salt form of the copper purpurin 7, an oxidized form of the copper purpurin 7, an oxidized form of the salt form of the copper purpurin 7, or a combination thereof,
(g) a copper rhodin g7, a salt form of the copper rhodin g7, an oxidized form of the copper rhodin g7, an oxidized form of the salt form of the copper rhodin g7, or a combination thereof, or
(h) a copper rhodochlorin, a salt form of the copper rhodochlorin, an oxidized form of the copper rhodochlorin, an oxidized form of the salt form of the copper rhodochlorin, or a combination thereof.

3. The method of claim 1 wherein the step of applying is only once per day and the method further comprises-repeating the step of applying for at least 83% of a number of days in a period of time, without skipping two consecutive days of the step of applying;
wherein the step of applying comprises applying a dose of 0.2 mg to 1.0 mg of the topical hair volumizing composition per 700 cm² of the human scalp; and
wherein the period of time is at least 70 days.

4. The method of claim 3 wherein (1) an average density of the terminal hairs increases by greater than 15% after the period of time compared to the average density prior to the period of time or (2) an average diameter of the terminal hairs increases by greater than 5% after the period of time compared to the average diameter prior to the period of time or (3) both; and
wherein the period of time is 90 days and the number of days is at least 75 days.

5. The method of claim 3 wherein an average density of the terminal hairs increases by greater than 25% after the period of time compared to the average density prior to the period of time;
wherein the one or more chelated copper chlorin compounds comprises the salt form of the copper chlorin e6 or the oxidized form of the salt form of the copper chlorin e6;
wherein the salt form of the copper chlorin e6 is trisodium copper chlorin e6;
wherein the one or more chelated copper chlorin compounds further comprises a copper rhodin g7, a salt form of the copper rhodin g7, an oxidized form of the copper rhodin g7, an oxidized form of the salt form of the copper rhodin g7, or a combination thereof; and
wherein the period of time is 180 days and the number of days is at least 150 days.

6. The method of claim 5 wherein an average diameter of the terminal hairs increases by greater than 15% after the period of time compared to the average diameter prior to the period of time.

7. The method of claim 1 wherein the carrier comprises at least 30% of an alcohol and at least 26.70% water, the percentages by weight of the topical hair volumizing composition.

8. The method of claim 1 comprising a further step of administering to a human an oral supplement comprising at least one hair benefit nutritional ingredient comprising an antioxidant, a vitamin, a mineral, a botanical extract, a marine extract, a protein, an amino acid, or a combination thereof.

9. The method of claim 1 further comprising a steps of applying a 6-piperidin-1-ylpyrimidine-2,4-diamine 3-oxide to the area of human scalp.

10. The method of claim 1 further comprising periodically repeating the step of applying over a period of time of at least 90 days; and
wherein a ratio of the terminal hairs to vellus hairs increases from about 5:1 prior to the period of time to about 9:1 after the period of time.

11. The method of claim 10 wherein the period of time is at least 180 days and wherein the ratio of the terminal hairs to the vellus hairs increases to about 20:1 after the period of time.

12-19. (canceled)

20. The method of claim 3 wherein (1) an average density of the terminal hairs increases by greater than 25% after the period of time compared to the average density prior to the period of time or (2) an average diameter of the terminal hairs increases by greater than 15% after the period of time compared to the average diameter prior to the period of time; and
wherein the period of time is 180 days.

21. The method of claim 1 further comprising a step of administering a 5-α reductase inhibitor, wherein the 5-α reductase inhibitor comprises one or more 4-azasteroid compounds.

22. The method of claim 2 wherein the salt form of the copper chlorin e6 is trisodium copper chlorin e6; and
wherein the one or more chelated copper chlorin compounds comprises the copper rhodin g7, the salt form of the copper rhodin g7, the oxidized form of the copper rhodin g7, the oxidized form of the salt form of the copper rhodin g7, or a combination thereof.

23. The method of claim 22 wherein the one or more chelated copper chlorin compounds comprises at least 80% by weight total of the salt form of the copper chlorin e6 or the oxidized form of the salt form of the copper chlorin e6 or a combination thereof.

24. The method of claim 23 further comprising repeating once per day the step of applying for at least 83% of a number of days in a period of time, without skipping two consecutive days of the step of applying;
wherein the step of applying comprises applying a dose of 0.2 mg to 1.0 mg of the topical hair volumizing composition per 700 cm² of the human scalp;
wherein the period of time is at least 90 days; and
wherein an average density of vellus hairs in the area of human scalp decreases by greater than 55% after the period of time compared to the average density prior to the period of time.

25. The method of claim 24 wherein the period of time is 180 days and wherein the average density of vellus hairs in the area of human scalp decreases by greater than 70% after the period of time compared to the average density prior to the period of time.

26. A method for increasing a density or a diameter of terminal hairs comprising a step of applying a topical hair volumizing composition comprising a carrier and 0.001% to 0.1% by weight of an active ingredient useful in increasing the density or the diameter of terminal hairs to an area of human scalp experiencing hair thinning resulting from aging;
wherein the active ingredient consists of one or more chelated copper chlorin compounds; and
wherein the one or more chelated copper chlorin compounds comprises at least 30% by weight of a copper isochlorin e4, a salt form of the copper isochlorin e4, an oxidized form of the copper isochlorin e4, an oxidized form of the salt form of the copper isochlorin e4, or a combination thereof.

27. The method of claim 26 wherein the one or more chelated copper chlorin compounds comprises the salt form of the copper isochlorin e4 or the oxidized form of the salt form of the copper isochlorin e4, or a combination thereof; and
wherein the salt form of the copper isochlorin e4 is disodium isochlorin e4;
wherein the one or more chelated copper chlorin compounds further comprises one or more of:
(a) a copper chlorin e4, a salt form of the copper chlorin e4, an oxidized form of the copper chlorin e4, an oxidized form of the salt form of the copper chlorin e4, or a combination thereof,
(b) a copper chlorin e6, a salt form of the copper chlorin e6, an oxidized form of the copper chlorin e6, an oxidized form of the salt form of the copper chlorin e6, or a combination thereof,
(c) a copper chlorin p6, a salt form of the copper chlorin p6, an oxidized form of the copper chlorin p6, an oxidized form of the salt form of the copper chlorin p6, or a combination thereof,
(d) a copper pheophorbide a, a salt form of the copper pheophorbide a, an oxidized form of the copper pheophorbide a, an oxidized form of the salt form of the copper pheophorbide a, or a combination thereof,
(e) a copper pyropheophorbide a, a salt form of the copper pyropheophorbide a, an oxidized form of the copper pyropheophorbide a, an oxidized form of the salt form of the copper pyropheophorbide a, or a combination thereof,
(f) a copper purpurin 7, a salt form of the copper purpurin 7, an oxidized form of the copper purpurin 7, an oxidized form of the salt form of the copper purpurin 7, or a combination thereof,
(g) a copper rhodin g7, a salt form of the copper rhodin g7, an oxidized form of the copper rhodin g7, an oxidized form of the salt form of the copper rhodin g7, or a combination thereof, or
(h) a copper rhodochlorin, a salt form of the copper rhodochlorin, an oxidized form of the copper rhodochlorin, an oxidized form of the salt form of the copper rhodochlorin, or a combination thereof.

28. The method of claim 27 wherein the step of applying is only once per day and wherein the method further comprises repeating the step of applying for at least 83% of a number of days in a period of time, without skipping two consecutive days of the step of applying;
wherein the step of applying comprises applying a dose of 0.2 mg to 1.0 mg of the topical hair volumizing composition per 700 cm² of the human scalp; and
wherein the period of time is at least 70 days.

29. The method of claim 28 wherein (1) an average density of the terminal hairs increases by greater than 15% after the period of time compared to the average density prior to the period of time or (2) an average diameter of the terminal hairs increases by greater than 5% after the period of time compared to the average diameter prior to the period of time or (3) both; and
wherein the period of time is at least 90 days and the number of days is at least 75 days.

30. The method of claim 29 wherein an average density of the terminal hairs increases by greater than 25% after the period of time compared to the average density prior to the period of time;
wherein the one or more chelated copper chlorin compounds comprises the salt form of the copper isochlorin e4 and the oxidized form of the salt form of the copper isochlorin e4;
wherein the period of time is at least 180 days and the number of days is at least 150 days.

31. The method of claim 30 wherein the one or more chelated copper chlorin compounds does not comprise the copper chlorin e6, the salt form of the copper chlorin e6, the oxidized form of the copper chlorin e6, or the oxidized form of the salt form of the copper chlorin e6.

32. The method of claim 1 further comprising a step of administering a retinoid.

33. The method of claim 9 wherein the carrier comprises 30-40% alcohol and 26.70-62.76% water, the percentages by weight of the topical hair volumizing composition.

34. The method of claim 21 wherein the carrier comprises 30-40% alcohol and 26.70-62.76% water, the percentages by weight of the topical hair volumizing composition.

35. The method of claim 7 wherein the carrier further comprises an antioxidant.

36. The method of claim 7 wherein the alcohol comprises SD alcohol, 190 proof.

37. The method of claim 36 wherein the carrier further comprises 2-10% by weight ethoxydiglycol.

38. The method of claim 37 wherein the carrier further comprises 5-20% butylene glycol.

39. The method of claim 1 wherein the carrier comprises water, denatured alcohol, ethoxydiglycol, and butylene glycol.

40. The method of claim 1 wherein the carrier comprises:
26.7-62.76% water;
30-40% denatured alcohol;
5-20% butylene glycol;
0.2-0.3% of phosphatidylcholine or lecithin;
0.01-0.1% of an antioxidant;
0.01-0.1% of a pH adjusting agent; and
wherein the percentages are by weight of the topical hair volumizing composition.

41. The method of claim 26 wherein the carrier comprises water, denatured alcohol, ethoxydiglycol, and butylene glycol.

42. The method of claim 37 wherein the carrier comprises:
26.7-62.76% water;
30-40% denatured alcohol;
5-20% butylene glycol;
0.2-0.3% of phosphatidylcholine or lecithin;
0.01-0.1% of an antioxidant;
0.01-0.1% of a pH adjusting agent; and
wherein the percentages are by weight of the topical hair volumizing composition.

43. A method for increasing a density or a diameter of terminal hairs comprising a step of applying once per day a topical hair volumizing composition comprising a carrier and 0.001% to 0.1% by weight of an active ingredient useful in increasing the density or the diameter of terminal hairs to an area of human scalp experiencing hair thinning resulting from aging; and
repeating the step of applying for at least 83% of a number of days in a period of time, without skipping two consecutive days of the step of applying;
wherein the step of applying comprises applying a dose of 0.2 mg to 1.0 mg of the topical hair volumizing composition per 700 cm² of the human scalp;
wherein the active ingredient comprises one or more chelated copper chlorin compounds; and
wherein the one or more chelated copper chlorin compounds comprises at least 75% by weight total of a copper chlorin e6, a salt form of the copper chlorin e6, an oxidized form of the copper chlorin e6, or an oxidized form of the salt form of the copper chlorin e6 or a combination thereof; and
wherein the period of time is at least 70 days.
